(19) United States
(12) Patent Application Publication
(10) Pub. No.: US 2010/0152770 A1
(43) Pub. Date: Jun. 17, 2010

(54) Truncal Tourniquet System & Method

(76) Inventor: David Spencer, San Antonio, TX (US)

Correspondence Address: STRASBURGER & PRICE, LLP, 901 MAIN STREET, SUITE 4400, DALLAS, TX, 75202, US

(21) Appl. No.: 12/638,519

(22) Filed: Dec. 15, 2009

Related U.S. Application Data
(60) Provisional application No. 61/201,801, filed on Dec. 15, 2008.
(60) Provisional application No. 61/214,692, filed on Apr. 27, 2009.

(51) Int. Cl. A61B 17/00

(52) U.S. Cl. 606/203

(57) ABSTRACT

A bleed out prevention system and method includes an adjustable length attachment belt assembly which is passed around the body of a human being suspected of having an injured or severed major blood vessel. The adjustable length attachment belt assembly positions a plunger plate assembly over a location where compressive force can be applied to a portion of the major blood vessel which has not been injured or severed. A compressive force application assembly then applies force which reduces the size of the lumen in the major blood vessel thereby restricting or stopping the flow of blood therethrough.

### CROSS REFERENCE TO RELATED APPLICATION

This application claims the benefit of Provisional U.S. Patent Application No. 61/201,801 filed Dec. 15, 2008 and Provisional U.S. Patent Application No. 61/214,692 filed Apr. 27, 2009

### STATEMENT REGARDING FEDERALLY FUNDED RESEARCH AND DEVELOPMENT

The invention described in this patent application was not the subject of Federally sponsored research or development.

### FIELD

The present invention pertains to a system and method for preventing hemorrhaging from the blood vessels of injured human beings; more particularly disclosed system and method enables the application of sufficient compressive force, as opposed to circumferential force, on an injured blood vessel to reduce the size of the lumen within the blood vessel and thereby restrict or minimize the out flow of blood when a portion of the blood vessel has been damaged or severed.

### BACKGROUND

In military conflicts, such as the ones involving the United States Armed Forces in both Iraq and Afghanistan, one of the most dangerous injuries to a soldier is an abdominal and/or truncal injury. This is because an abdominal and/or truncal injury often damages or severs a major internal blood vessel. The result is uncontrolled hemorrhaging.

Because of the location of the injury, it may not be possible to place a tourniquet on the injured person; however, the application of compressive force from a selected location on the exterior of the injured person's body may reduce the blood flow out of the body. More particularly, such injuries occur in compressible but not tourniqueable parts of the body. Unless compressive force on these internal injuries is applied quickly, there is a danger that the wounded soldier will “bleed out” or simply lose the blood needed to sustain life.

While soldiers typically wear body armor to protect themselves from small arms fire, there are usually some small openings between some sections of the body armor. Such openings in armor protection may be present in the groin area, the armpit area and in the neck area. Accordingly, enemy soldiers are instructed to target the locations of an opening in the body armor to endeavor to cause a parenchymal wound in an unprotected area of a soldier's body. Such parenchymal wounds lead to either severe damage to or the severing of a major blood vessel. Damage to a major blood vessel typically causes a wounded soldier to hemorrhage and eventually die. A vivid portrayal of such an incident appeared in the movie “Blackhawk Down.”

Because much of the bodies of modern soldiers are protected from small arms fire by armor, the bullet strikes that were fatal in World War II, the Korean Conflict, and in Viet Nam are no longer as fatal as they once were. In prior combat, soldiers died from bullet wounds before ever losing their blood. Thus, a whole new challenge has been presented. That challenge is to treat damage to a major blood vessel for a solder who has survived multiple hits from small arms fire and would have died in prior years.

Similarly, many people involved in automobile accidents suffer abdominal and/or truncal or other injuries which sever major blood vessels. If those injuries which sever major blood vessels are not properly treated the person injured in an automobile accident will hemorrhage and die from loss of blood.

What is needed in the art is a system and method which will easily and quickly enable sufficient compressive force to be placed on that internal portion of the body of a wounded soldier or an injured person which will restrict or minimize the outflow of blood so that blood flow through injured blood vessels is restricted or stopped. Such restriction or minimization of the outflow of blood will provide the time needed for emergency medical help to arrive and can stay in place while the injured person is being transported to a medical care facility where definitive surgical care is available.

### SUMMARY

The system and method of the disclosed invention enables the easy and quick application of sufficient compressive force on those untourniqueable areas of the body of a wounded soldier or of an injured person through which large blood vessels pass. Such compressive force causes the openings through the large blood vessels to be reduced in size so that flow of blood therethrough will be restricted or minimized until emergency medical help arrives.

The system and method of the present invention includes three parts. The first part is an adjustable length attachment belt assembly. The adjustable length attachment belt assembly positions the second part of the disclosed invention, the plunger plate assembly, over on area on the body of an injured person where compressive force can be applied to an uninjured portion of a large blood vessel proximal to the injured portion of the body. The area where compressive force is applied is between the heart and the damaged portion of the major blood vessel. The plunger plate assembly enables the third part of the invention, the compressive force application assembly, to apply sufficient compressive force on a large blood vessel to reduce the size of the lumen through the blood vessel. Such compressive force restricts or minimizes the flow of blood through the blood vessel. The compressive force application assembly may include a body contact foot which conforms to the countour of the body where compressive force is applied.

### BRIEF DESCRIPTION OF THE DRAWING FIGURES

A still better understanding of the truncal tourniquet prevention system and method of the present invention may be had by reference to the drawing figures, wherein:

FIG. 1A is a perspective view of the bottom of a first embodiment of the invention;

FIG. 1B is a perspective view of the top of a second embodiment of the invention;

FIG. 2 is an enlarged side perspective view of the second and preferred embodiment of the invention;

FIG. 3 is a front perspective view of the application of the first embodiment of the invention to the abdomen of a human being;

FIG. 4 is a front perspective view of the application of the second embodiment of the invention to the neck of a human being;

FIG. 5 is a perspective view of the application of the second embodiment of the invention to the armpit of a human being.

### DESCRIPTION OF THE EMBODIMENTS

The disclosed invention was created in response to the growing need for a device which is easily usable in a combat environment, particularly a combat environment such as experienced in Iraq and Afghanistan where many deaths are caused by uncontrolled hemorrhaging from an injured or severed major blood vessel. In Iraq and Afghanistan most injuries are caused by improvised explosive devices. Unfortunately, such encounters often take place in remote locations where medical aid will not be quickly available.

The explosion of an improvised explosive device is particularly insidious as it is designed to create a small explosion near a vehicle. The blast and the pressure wave from the improvised explosive device exposes the occupants of the vehicle to penetrating traumatic injuries from various parts of the vehicle as the vehicle disintegrates and eventually catches fire. Because of the multiple sources of potential penetrating traumatic injuries there is a chance that the damage caused by the disintegration of the vehicle will create an injury which will sever a major blood vessel of one of the vehicle's occupants. While the injuries to the occupants of the vehicle may not be fatal, the severing of a major blood vessel often creates a pathway through which blood can exit the body without restraint. In such cases the life of a soldier is lost not from injuries sustained during the disintegration of the vehicle, but from simply hemorrhaging through a severed major blood vessel before emergency medical help is able to arrive and stop the bleeding.

Those of ordinary skill in the art will understand that damage to and explosions of vehicles or other equipment can also happen in a civilian setting. In such civilian setting there is also the possibility of an occupant of a vehicle may hemorrhage through a damaged or severed major blood vessel before emergency medical help can arrive.

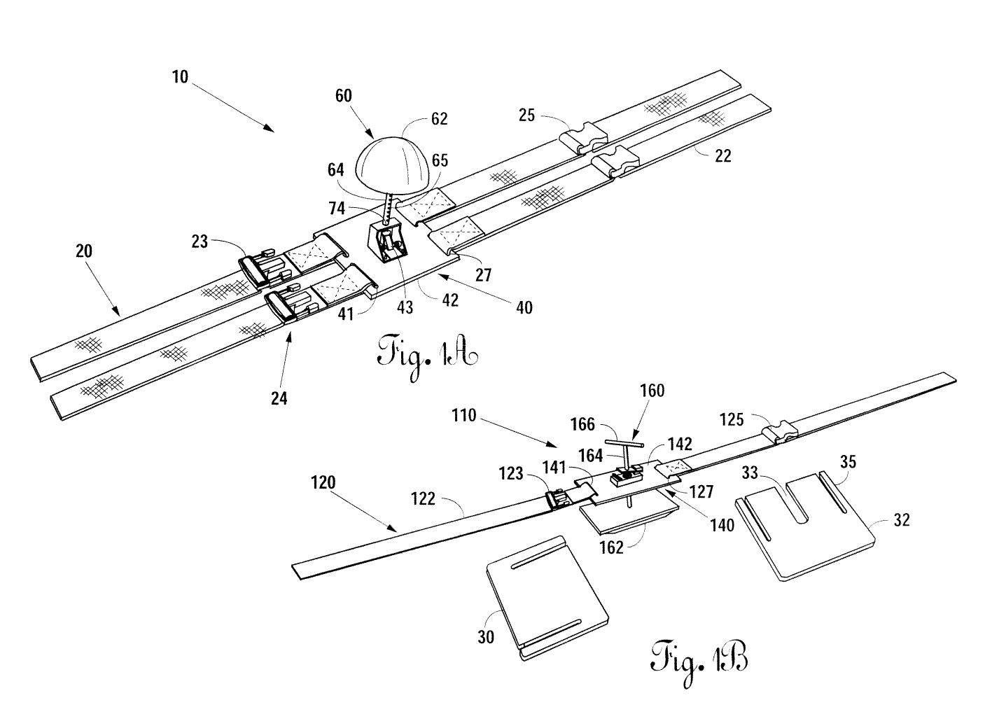

To meet the need for a system and method that can be quickly and easily used for preventing the hemorrhaging of a human being through a damaged for severed major blood vessel the disclosed invention has been created. Specifically, the disclosed invention 10, 110 includes three portions 20, 40, and 60 as shown in FIG. 1A and portions 120, 140 and 160 as shown in FIG. 1B.

Referring to the embodiment 10 shown in FIG. 1A, the first assembly is the adjustable length attachment belt assembly 20. The adjustable length attachment belt assembly 20 includes a belt or belts 22 and a positionable clip assembly 24. It is the belt 22 which is passed around the body of the injured person to hold the other two portions 40, 60 of the invention 10 in place.

Securing the belt 22 around the body of the injured person are first and second positionable clip portions 23, 25. The first and second positionable clip portions 23, 25 may be moved with respect to belt 22 and connect with one another to form clip assembly 24. The adjustable length attachment belt assembly 20 provides the foundation for the compressive forces which will eventually be used against the non-inured portion of a severed blood vessel to collapse the lumen within the non-injured portion of the severed blood vessel to restrict or minimize the flow of blood therethrough.

As shown in the second embodiment 110 in FIG. 1B, the adjustable length attachment belt assembly 120 bears the same relationship to assemblies 140 and 160 as in the first embodiment 10. Further, a plate 30, is shown with the second embodiment 110 to enable distribution of the compressive forces from the adjustable length attachment belt assembly 120 against the body of the injured person. For example, in both the first and second embodiments 10, 110, a plate is placed against the back or side of the injured person when pressure is to be exerted on the non-injured portion of a major blood vessel passing through the abdomen. While plate 30 is shown with the second embodiment 110, those using either the first or second embodiments 10, 110 may use a piece of wood, a piece of metal, a manual, a book or even a flat rock under belts 22, 122 to distribute the forces against the body of the injured person. Also shown in FIG. 1B is an anti-wobble plate 32 which may be used to stabilize the surface applying the compressive force to the body of the inured person. The use of the anti-wobble plate 32 will be explained in greater detail below.

The second assembly is the plunger plate assembly 40, 140. The plunger plate assembly 40, 140 is connected to the adjustable length attachment belt assembly 20, 120. Such connection may be effected by passing the belts 22 or belt 122 through the slots 41 in the plate portion 42, 142 and attaching the belts 22 or belt 122 back on themselves to form a loop 27, 127 connecting the belts 22 or belt 122 to the plate 42, 142. It is the plunger plate assembly 40, 140 which is positioned in the desired location near the non-injured portion of a major blood vessel by the adjustable length attachment belt assembly 20, 120. As indicated above the adjustable length attachment belt assembly 20, 120 becomes the foundation for the compressive forces to be used to push against the non-injured portion of a major blood vessel. More specifically, the adjustable length attachment belt assembly 20, 120 becomes the foundation for the plunger plate assembly 40, 140 which enables the application of compressive forces against the non-injured portion of major blood vessel by the compressive force application assembly 60, 160.

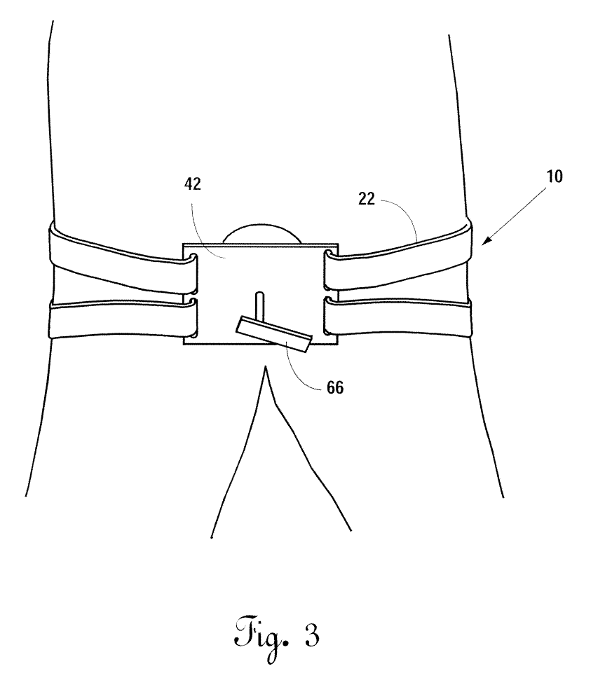

The compressive force application assembly 60, 160 is the third portion of the invention 10, 110. It is the compressive force application assembly 60, 160 which actually pushes against the non-injured portion of a major blood vessel to reduce the size of the lumen which restricts or minimizes the blood flow therethrough. The parts of the compressive force application assembly 60, 160 are a body contact foot 62, 162, a shaft 64, 164 which extends outwardly from the body contact foot 62, 162 and a handle 166 (handle obscured in FIG. 1A but shown in FIG. 3) at the opposite end of the shaft 64. It is the shaft 64, 164 which passes through a hole in the plunger plate assembly 40, 140 to allow pressure to be applied against a major blood vessel. The compressive force application assembly 60, 160 is held in position and restricted in movement by the plunger plate assembly 40, 140 as the body contact foot 62, 162 actually applies sufficient compressive force to the body of the injured person when the shaft 64, 164 is moved through the plate 42, 142 to restrict or minimize blood flow through the lumen of a large blood vessel.

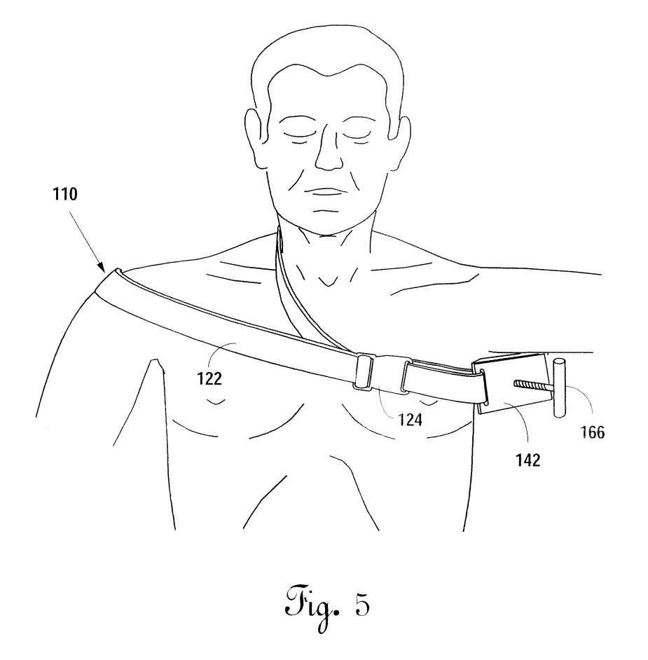

Mechanical connection between the shaft 64, 164 and the plunger plate assembly 40, 140 may incorporate several types of different systems well known to those of ordinary skill in the art. For example, the shaft 64, 164 may be externally threaded (shown in FIG. 5) and caused to pass through an internally threaded device such as a nut. The nut may be welded or mechanically affixed to the plate 42, 142 which makes up part of the plunger plate assembly 40, 140. Those of ordinary skill in the art will also understand that the shaft 64, 164 may be hinged or telescoped to reduce its length. For example, the shaft 64, 164 may be segmented with an elastic cord passing therethrough so that segments of the shaft 64, 164 may be folded back on one another when not in use and extended when in use. Alternatively, there may be a ratchet type connection 74 as shown in FIG. 1A wherein the shaft 64 may pass through the plate 42 portion of the plunger plate assembly 40 while engaging a keeper 43 which allows the shaft 64 to pass in one direction and not in the opposite direction. Release of the shaft 64 with a ratchet type connection 74 may be obtained from placing a quarter or half turn on the handle 66 at the top end of shaft 64. This quarter or half turn turns the shaft 64 thereby moving the ratchet teeth 65 away from the keeper 43 and allowing the shaft 64 to pass easily therethrough.

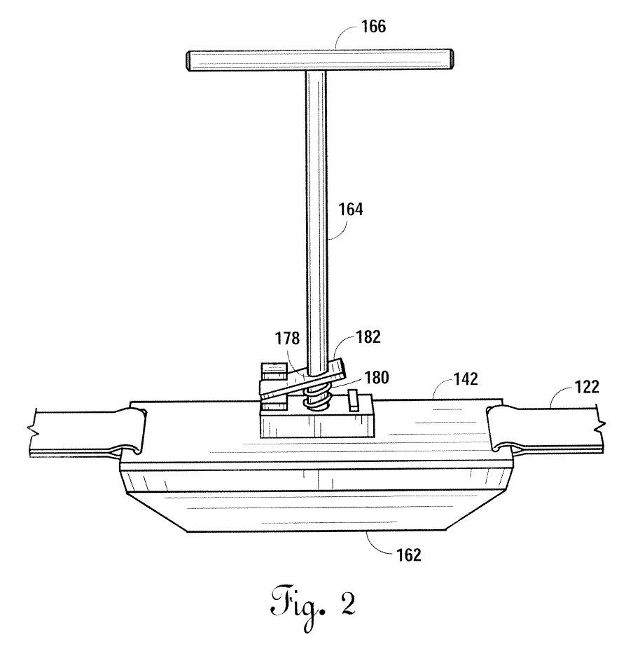

In FIG. 1B, the connection between the shaft 164 and the plate portion 142 of the plunger plate assembly 40 includes a spring loaded friction connection 76. The spring loaded friction connection 76, shown in more detail in FIG. 2, may be used. Herein the shaft 64 is held in place by the friction against the sides of a hole 178 in a spring 180 biased piece of metal 182 angled against the exterior surface of the shaft 164.

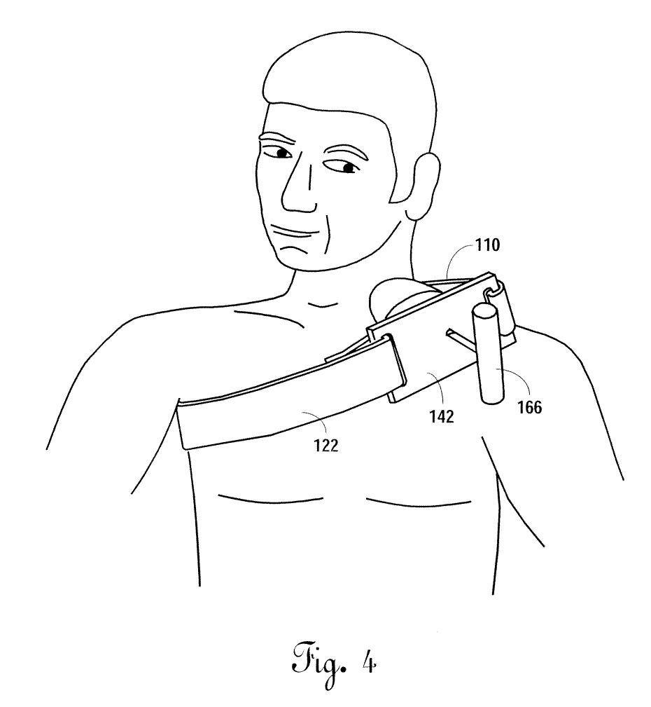

It has been found that different shapes of a body contact foot 62, 162 may be used. For example, a substantially semi-spherical or ball shape may be used as shown in FIG. 1A. Alternatively, a body contact foot 162 may have a V-shape or a triangular cross section as shown in FIG. 1B and in FIG. 2. Still other shapes of a body contact foot, such as the parabolic shape shown in FIG. 4, may be used without departing from the scope of the disclosed invention. The body contact foot may also be made to be easily shapeable to conform to the contour of the body to which pressure is to be applied.

Those of ordinary skill in the art will understand that some designs of the body contact foot may roll or wobble when compressive force is applied. To prevent such roll or wobble, a plate 32 as shown in FIG. 1B may be used. The slot 33 is placed around shaft 64, 164 and the legs 35 are placed through loops 27, 127 in the belts 22, 122.

A still better understanding of the disclosed invention may be had from a description of its operation as depicted in FIGS. 3, 4 and 5. Specifically, FIG. 3 shows embodiment 10 from FIG. 1A in place against the abdomen of an injured person. As indicated above, in some circumstances a board or flat surface (not shown) be placed against the body of the injured person to distribute forces. In FIG. 4, the embodiment 110 from FIG. 1B is in place against the side of an injured person's neck. And, in FIG. 5, the embodiment 110 from FIG. 1B is in place against an injured person's armpit.

After an occupant of a vehicle involved in an accident or an explosion or a person involved in some other type of mishap is perceived to be losing blood at a rapid rate because a major blood vessel, such as femoral artery, has been either injured or severed, there is a critical need to prevent the continued flow of blood through the injured or severed major blood vessel. Because it is often impossible to gain direct access to that portion of a major blood vessel which is either injured or severed, it then becomes necessary to close the major blood vessel to the flow of blood therethrough by applying compressive force on a portion of the major blood vessel which retains some structural integrity and is closer to the heart of the injured person. Since many major blood vessels, pass just under the skin at various locations in the body, it is oftentimes possible to apply sufficient compressive point pressure, as opposed to the circumferential force of a tourniquet, on a major blood vessel to collapse the lumen within the blood vessel by applying compressive force to the external surface of the body of the injured person. For example, compressive force may be placed on the abdomen, as shown in FIG. 3, the side of the neck as shown in FIG. 4, or the armpit as shown in FIG. 5. Such compressive force will either stop or diminish the flow of blood through the severed or injured blood vessel long enough for medical help to arrive.

To establish compressive force against the abdomen, the armpit or the side of the neck of an injured person it is first necessary to locate the most effective spot at which compressive force may be applied to collapse or reduce the size of the lumen through the injured or severed blood vessel. Once the most effective spot has been located, the next step is to establish a foundation for the application of compressive force to put compressive force on the injured or severed blood vessel.

The construction of the foundation for the application of compressive force begins with the passage of the belt portion of the adjustable length attachment belt assembly 20, 120 around the body of the injured person. Specifically, a portion of the injured person's body on the opposite of the body from where compressive force is to be applied serves as the foundation for the application of compressive force. Passage of the belt portion 22, 122 around that portion of the body opposite from where compressive force is to be applied begins the construction of the foundation.

For the abdomen, as shown in FIG. 3, the foundation is constructed by passing the belt portion 22, 122 of the adjustable length attachment belt assembly 20, 120 around the back of the injured person. For compressive pressure against the side of the neck or against the armpit, the adjustable length attachment belt assembly is passed around the chest as shown in FIG. 4 and FIG. 5 respectively.

Following the passage of the belt portion 22, 122 of the adjustable length attachment belt assembly 20, 120 around the body of the injured person and the securement of the first and second clip portions 23, 25, 123, 125 one to another to create the clip assembly 24, 124, the plunger plate assembly 40, 140 is positioned over the location where compressive force is to be applied against that portion of a major blood vessel still having sufficient integrity so that its lumen may be temporarily collapsed to prevent or restrict the flow of blood therethrough. With the plunger plate assembly 40, 140 in place over the desired location, the clip portions 23, 25, 123, 125 on the ends of the adjustable length attachment belt assembly 20, 120 are attached to one another to create clip assembly 24, 124 and the adjustable length attachment belt assembly 20 is tightened around the injured person.

With the plunger plate assembly 40, 140 in place, all that remains is to apply compressive force directed toward the body of the injured person on the handle portion 66, 166 of the compressive force application assembly 60, 160. Such force will cause the shaft 64, 164 moving through the plate portion 42, 142 of the pressure plate assembly 40, 140 to cause the body contact foot 62, 162 to apply pressure to the major blood vessel. As previously indicated, the shaft 64, 164 may be moved by threadable engagement (shown in FIG. 5), by a ratchet engagement (shown in FIG. 1A) or passage through a friction keeper (shown in FIG. 1B and FIG. 2).

The device of the present invention is kept in place long enough to permit medical personnel to arrive and either treat the site of the injured or severed blood vessel or replace the blood being lost at both the location of explosion or accident and to continue to control hemorrhaging during transport to more definitive surgical care.

Release of the device of the present invention from the injured person is accomplished by counter rotation of the threadable engagement of the shaft with the plate portion of the plunger plate assembly, release of the ratchet teeth from the keeper or release of the frictional forces on the side of the shaft.

While the present invention has been disclosed according to its preferred and alternate embodiments, yet other modifications and embodiments will become apparent to those of ordinary skill in the art. Such modifications and embodiments shall be included within the scope and meaning of the appended claims.

What is claimed is:

1. A system for reducing the size of the lumen of a damaged blood vessel in a human being, said system comprising:
an adjustable length attachment belt assembly;
a plunger plate assembly, said plunger plate assembly being positionable by said adjustable attachment belt assembly over a blood vessel;
a compressive force application assembly constructed and arranged to move through said plunger plate assembly a sufficient distance to apply pressure to a blood vessel;
whereby said pressure on the blood vessel will reduce the size of the lumen through the blood vessel and thereby restrict or reduce the flow of blood therethrough.

2. The system for reducing the size of the lumen of a damaged blood vessel as defined in claim 1 wherein said adjustable length attachment belt assembly includes a first positionable clip portion on one end and a second positionable second clip portion on the other end, said first and second positionable clip portions enabling attachment with one another.

3. The system for reducing the size of the lumen of a damaged blood vessel as defined in claim 1 wherein said adjustable length attachment belt assembly includes a plate for placement against the back of the human being when compressive force is to be applied to the abdomen of the human being.

4. The system for reducing the size of the lumen of a damaged blood vessel as defined in claim 1 wherein said compressive force application assembly includes: a body contact foot, a shaft extending outwardly from said body contact foot, and a handle at the opposite end of said shaft from said body contact foot.

5. The system for reducing the size of the lumen of a damaged blood vessel as defined in claim 4 wherein said plunger plate assembly includes means for controlling the position and restricting the movement of said shaft therethrough.

6. The system for reducing the size of the lumen of a damaged blood vessel as defined in claim 5 wherein said means for controlling the position and restricting the movement of said shaft therethrough is selected from a group including: threadable engagement, a releasable ratchet connection and frictional engagement.

7. The system for reducing the size of the lumen of a damaged blood vessel as defined in claim 4 wherein said body contact foot is semi-spherical.

8. The system for reducing the size of the lumen of a damaged blood vessel as defined in claim 4 wherein said body contact foot has a substantially triangular cross section.

9. The system for reducing the size of the lumen of a damaged blood vessel as defined in claim 8 further including an anti-wobble plate.

10. A method for reducing the size of the lumen of a damaged blood vessel in a human being by applying pressure to the exterior surface of said human being, said method comprising the steps of:
passing the belt portion of an adjustable length attachment belt assembly around the body of the human being to contact that portion of the human being opposite the side of the human being where pressure is to be applied to the exterior surface of the human being;
locating a plunger plate assembly over the site where pressure is to be applied to the exterior surface of the human being;
moving a compressive force application assembly through said plunger plate assembly to apply pressure to the exterior surface of the human being.

11. The method as defined in claim 10 wherein said adjustable length attachment belt assembly includes a first positionable clip portion on one end and a second positionable clip portion on the other end, said first and second positionable clip portion enabling attachment with one another.

12. The method as defined in claim 10 wherein said adjustable length attachment belt assembly includes a rigid plate for placement against the back of the human being when pressure is to be applied against the abdomen of the human being.

13. The method as defined in claim 10 wherein said compressive force application assembly includes: a body contact foot, a shaft extending outwardly from said body contact foot, and a handle at the opposite end of said shaft from said body contact foot.

14. The method as defined in claim 13, wherein said plunger plate assembly includes means for controlling the position of and restricting the movement of said shaft therethrough.

15. The method as defined in claim 14 wherein said means for controlling the position and restricting the movement of said shaft therethrough is selected from a group including: threadable engagement, a releasable ratchet connection and frictional engagement.

16. The method as defined in claim 10 further including the use of an anti-wobble plate with said plunger plate assembly.
